(19) United States
(12) Patent Application Publication
(10) Pub. No.: US 2026/0364373 A1
(43) Pub. Date: Nov. 19, 2026

(54) CATHETER, AND CATHETER SET AND LIGHT-DIFFUSING DEVICE INCLUDING CATHETER

(71) Applicant: FURUKAWA ELECTRIC CO., LTD., Tokyo (JP)

(72) Inventors: Kumi KAWASHIMA, Tokyo (JP); Junichi HASEGAWA, Tokyo (JP); Hideaki HASEGAWA, Tokyo (JP); Kyosuke YAMAUCHI, Tokyo (JP); Keisuke TAKE, Tokyo (JP); Takuya HASEGAWA, Tokyo (JP)

(73) Assignee: FURUKAWA ELECTRIC CO., LTD., Tokyo (JP)

(21) Appl. No.: 19/470,510

(22) PCT Filed: Mar. 29, 2024
(86) PCT No.: PCT/JP2024/013101

§ 371 Date: Sep. 29, 2025

(30) Foreign Application Priority Data
Mar. 30, 2023 (JP) 2023-055250

(51) Int. Cl. A61N 5/06; A61N 5/067

(52) U.S. Cl. CPC A61N 5/062; A61N 5/0601; A61N 5/067; A61N 2005/063; A61N 2005/0632; A61N 2005/0665

(57) ABSTRACT

A catheter is configured so that when used with an optical fiber inserted therein, light can be efficiently emitted toward a specific direction of a lateral surface. A catheter set and a light-diffusing device may include the catheter. The catheter that includes a catheter body having a base end and a tip end. A light-blocking element is provided to the catheter body.

### TECHNICAL FIELD

The present invention relates to a catheter used as a medical instrument, and to a catheter set and a light diffusion device including the catheter.

### BACKGROUND ART

Conventionally known examples of a light diffusion device (diffuser) include an optical fiber including: a core positioned on a center side in a radial direction; and a cladding positioned on an outer peripheral side of the core, in which the laser light incident on a proximal end portion of the optical fiber is emitted from a distal end portion and an outer peripheral surface on a distal end side of the optical fiber (see, for example, Patent Document 1). The optical fiber of the conventional light diffusion device includes: a light transmission section that transmits laser light incident on the proximal end portion; and a light emission section that emits the laser light transmitted through the light transmission section at the distal end side.

In photodynamic therapy (PDT) and photoimmunotherapy (PIT), which are cancer treatments using light, a diffuser is inserted into a catheter, and the catheter is inserted into a body for light irradiation. The diffuser emits light laterally from the distal end portion of the optical fiber, and normally emits light in an omnidirectional manner; therefore, there is concern that the light may irradiate not only cancer cells but also healthy cells.

### CITATION LIST

### Patent Document

Patent Document 1: Japanese Unexamined Patent Application (Translation of PCT Application), Publication No. 2020-500575

### DISCLOSURE OF THE INVENTION

### Problems to be Solved by the Invention

Accordingly, one of the objects of the present invention is to provide a catheter capable of efficiently emitting light in a specific lateral direction, as well as a catheter set and a light diffusion device including the catheter, when an optical fiber such as a diffuser is inserted and used.

### Means for Solving the Problems

According to one aspect of the present invention, a catheter includes: a catheter body including a proximal end portion and a distal end portion; and a light shielding element provided on the catheter body.

In the catheter according to one aspect of the present invention, the light shielding element may be in contact with an inner peripheral surface or an outer peripheral surface of the catheter body.

In the catheter according to one aspect of the present invention, a balloon may be provided outside the catheter body including the light shielding element.

In the catheter according to one aspect of the present invention, the light shielding element may be made of metal.

In the catheter according to one aspect of the present invention, the light shielding element may be formed of a reflective resin.

In the catheter according to one aspect of the present invention, the light shielding element may be provided in an arc shape about an axis extending from the proximal end portion to the distal end portion of the catheter body.

In the catheter according to one aspect of the present invention, the light shielding element may be provided over the entire circumference about an axis extending from the proximal end portion to the distal end portion of the catheter body, and may include an opening on a lateral surface in a predetermined direction. In this case, the catheter body may have a property of being non-transmissive to light, a region in which the catheter body is not present may be provided,
the region in which the catheter body is not present may correspond to the opening, and
a position in an axial direction may be substantially the same as the region in which the catheter body is not present, and a region in which the catheter body is present may correspond to the light shielding element.

According to one aspect of the present invention, a catheter set includes: the catheter according to one aspect of the present invention; and
an optical fiber including a proximal end portion and a distal end portion and configured to emit light incident on the proximal end portion from an outer peripheral surface on a distal end side.

According to one aspect of the present invention, a light diffusion device includes: the catheter according to one aspect of the present invention; and
an optical fiber including a proximal end portion and a distal end portion and configured to emit light incident on the proximal end from an outer peripheral surface on a distal end side. The optical fiber is inserted into the catheter, such that the respective proximal end portions are aligned on the same side, and the respective distal end portions are aligned on the same side, and
in an axial direction extending from the proximal end portion to the distal end portion, a light emission position on the distal end side of the optical fiber overlaps with a position of the light shielding element of the catheter.

In the light diffusion device according to one aspect of the present invention, the light shielding element is provided over the entire circumference about an axis extending from the proximal end portion to the distal end portion, and includes an opening on a lateral surface in a predetermined direction, and
the optical fiber may be movable relative to the catheter in the axial direction.

In this case, a one-side clearance between the catheter body and the optical fiber may be 5% or more of an outer diameter of the optical fiber, and
a surface treatment for reducing friction may be applied to at least one or both of an inner peripheral surface of the catheter body or an outer peripheral surface of the optical fiber.

In the light diffusion device according to one aspect of the present invention in which the optical fiber is movable relative to the catheter in the axial direction, a stopper may be disposed inside the distal end portion of the catheter body.

In the light diffusion device according to one aspect of the present invention in which the optical fiber is movable relative to the catheter in the axial direction, a scale capable of checking an amount of movement of the optical fiber relative to the catheter body may be provided near the proximal end portion of the optical fiber.

In the light diffusion device according to one aspect of the present invention in which the optical fiber is movable relative to the catheter in the axial direction, a tubular glass cap with a sealed distal end may be attached so as to cover a light emission position on the distal end side of the optical fiber.

The light diffusion device according to one aspect of the present invention further include an endoscope, and the light shielding element may be in direct or indirect contact with a metal portion of the endoscope.

### Effects of the Invention

According to one aspect of the present invention, it is possible to provide a catheter capable of efficiently emitting light in a specific lateral direction, as well as a catheter set and a light diffusion device including the catheter, when an optical fiber such as a diffuser is inserted and used.

### BRIEF DESCRIPTION OF THE DRAWINGS

FIG. 1 is a longitudinal sectional view of a light diffusion device including a catheter according to a first embodiment as an exemplary aspect of the present invention, and is a cross-sectional view taken along the line C-C in FIG. 2;

FIG. 2 is a transverse sectional view near a distal end portion of the light diffusion device including the catheter according to the first embodiment, and is a cross-sectional view taken along the line A-A in FIG. 1;

FIG. 3 is a longitudinal sectional view of a modified example of the light diffusion device including the catheter according to the first embodiment, and is a cross-sectional view taken along the line E-E in FIG. 4;

FIG. 4 is a transverse sectional view of the modified example of the light diffusion device including the catheter according to the first embodiment, and is a cross-sectional view taken along the line D-D in FIG. 3;

FIG. 5 is a longitudinal sectional view of a light diffusion device including a catheter according to a second embodiment as an exemplary aspect of the present invention;

FIG. 6 is a longitudinal sectional view of a light diffusion device including a catheter according to a third embodiment as an exemplary aspect of the present invention;

FIG. 7 is a longitudinal sectional view of a light diffusion device including a catheter according to a fourth embodiment as an exemplary aspect of the present invention, and is a cross-sectional view taken along the line G-G in FIG. 8;

FIG. 8 is a transverse sectional view near a distal end portion of the light diffusion device including the catheter according to the fourth embodiment, and is a cross-sectional view taken along the line F-F in FIG. 7;

FIG. 9 is a front view of a light diffusion device including a catheter according to a fifth embodiment as an exemplary aspect of the present invention;

FIG. 10 is a longitudinal sectional view of the light diffusion device including the catheter according to the fifth embodiment, viewed from the right side in FIG. 9, and is a cross-sectional view taken along the line I-I in FIG. 11;

FIG. 11 is a transverse sectional view of the light diffusion device including the catheter according to the fifth embodiment, as viewed from the right side in FIG. 9, and is a cross-sectional view taken along the line H-H in FIG. 10;

FIG. 12 is a longitudinal sectional view of a light diffusion device illustrating an example of use of the catheter according to the fifth embodiment, illustrating a state in which an irradiation area is large (irradiation length is long) ;

FIG. 13 is a longitudinal sectional view of a light diffusion device illustrating an example of use of the catheter according to the fifth embodiment, illustrating a state in which the irradiation area is small (irradiation length is short) as compared to the state in FIG. 12;

FIG. 14 is a transverse sectional view of the catheter according to the fifth embodiment, and is a cross-sectional view taken along the line S-S in FIG. 12;

FIG. 15 is a longitudinal sectional view illustrating a modified example in which a stopper is disposed in the light diffusion device including the catheter according to the fifth embodiment;

FIG. 16 is a schematic view illustrating a state in which a glass cap is attached to the optical fiber;

FIG. 17 is a longitudinal sectional view of a light diffusion device including a catheter according to a sixth embodiment as an exemplary aspect of the present invention, and is a cross-sectional view taken along the line K-K in FIG. 18;

FIG. 18 is a transverse sectional view near a distal end portion of the light diffusion device including the catheter according to the sixth embodiment, and is a cross-sectional view taken along the line J-J in FIG. 17;

FIG. 19 is a longitudinal sectional view of a light diffusion device including a catheter according to a seventh embodiment as an exemplary aspect of the present invention, and is a cross-sectional view taken along the line N-N in FIG. 20;

FIG. 20 is a transverse sectional view near a distal end portion of the light diffusion device including the catheter according to the seventh embodiment, and is a cross-sectional view taken along the line M-M in FIG. 19;

FIG. 21 is a front view of a light diffusion device including a catheter according to an eighth embodiment as an exemplary aspect of the present invention;

FIG. 22 is a longitudinal sectional view of the light diffusion device including the catheter according to the eighth embodiment, viewed from the right side in FIG. 21, and is a cross-sectional view taken along the line R-R in FIG. 23; and

FIG. 23 is a transverse sectional view of the light diffusion device including the catheter according to the eighth embodiment, viewed from the right side in FIG. 21, and is a cross-sectional view taken along the line Q-Q in FIG. 22.

### PREFERRED MODE FOR CARRYING OUT THE INVENTION

### Catheter and Light Diffusion Device

Hereinafter, the catheter and the light diffusion device including the catheter according to eight embodiments as exemplary aspects of the present invention will be described in detail with reference to the drawings.

### First Embodiment

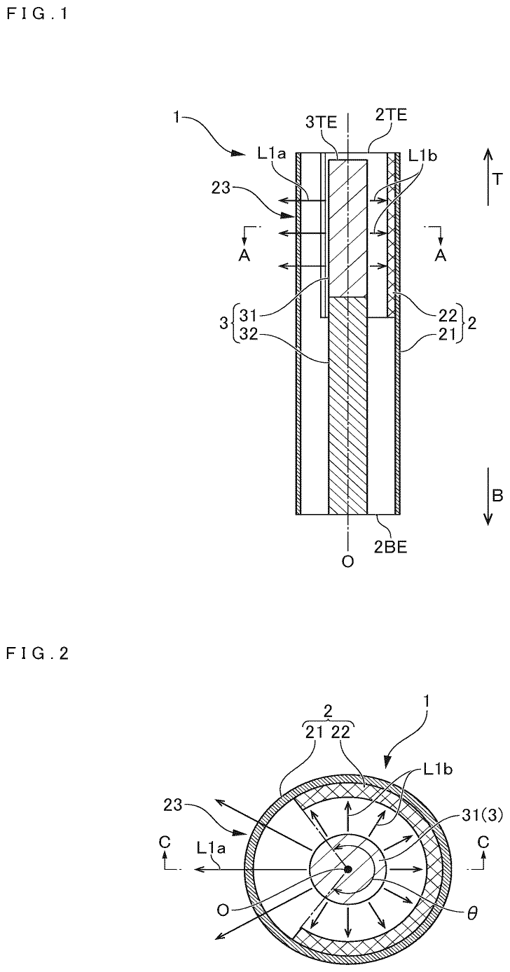

FIG. 1 is a longitudinal sectional view of a light diffusion device 1 including a catheter 2 according to the first embodiment; and FIG. 2 is a transverse sectional view near a distal end portion of the light diffusion device 1.

FIG. 1 is a cross-sectional view taken along the line C-C in FIG. 2; and FIG. 2 is a cross-sectional view taken along the line A-A in FIG. 1.

In the longitudinal sectional view in FIG. 1, a proximal side of the catheter 2 is indicated by the arrow B, a distal side is indicated by the arrow T, a distal end portion is indicated by the reference sign 2TE, and a proximal end portion is indicated by the reference sign 2BE. Note that the longitudinal sectional view in FIG. 1 is a schematic diagram, and the catheter 2 actually has a more elongated and slender shape (the same applies to the longitudinal sectional views in the other embodiments and their modifications hereinafter).

The light diffusion device 1 includes the catheter 2 according to the present embodiment and an optical fiber 3.

The catheter 2 includes a tubular catheter body 21 and a light shielding element 22 provided on the catheter body 21. The light shielding element 22 is attached in contact with an inner peripheral surface of the catheter body 21.

Meanwhile, the optical fiber 3 includes a proximal end portion (not illustrated) and a distal end portion 3TE, and radially emits light, incident on the proximal end portion, as light L1a and L1b from a lateral surface on the distal side T. In the schematic longitudinal sectional view in FIG. 1, the proximal side B of the optical fiber 3 is cut together with the catheter 2, but in practice, the optical fiber 3 extends from the proximal end portion 2BE of the catheter 2 and is connected at the proximal end portion (not illustrated) to a laser oscillator (not illustrated) serving as a light source for generating laser light (the same applies to the longitudinal sectional views of other embodiments and their modifications hereinafter).

The catheter body 21 is a product generally marketed or used as a medical catheter. The term “catheter” typically refers to a medical tube in the form of either a cylinder having a diameter of 20 mm or less, or a rectangular prism having a side length of 20 mm or less. The so-called “lumen” or “lumen catheter” is also included in the scope of the catheter in the present invention.

As illustrated in FIG. 2, the light shielding element 22 is provided in an arc shape with a central angle θ about an axis O (hereinafter also simply referred to as “axis O”, the same applies to other embodiments and their modifications hereinafter) extending from the proximal end portion 2BE to the distal end portion 2TE of the catheter body 21. In the schematic drawing of FIG. 1 (and other longitudinal sectional views), the axis O is depicted as a straight line, but since the catheter 2 is a flexible tubular member, the axis O may be curved in accordance with a degree of bending.

The light shielding element 22 is a member that blocks the light Lib from leaking out of the catheter body 21. Specifically, the light L1b is emitted radially from the optical fiber 3 in circumferential directions (directions within a range of a central angle θ) other than the direction of the light L1a that exits to the outside through the opening 23.

In the present embodiment, the central angle e is set, for example, to 250°. The central angle θ is not particularly limited, but may be appropriately set depending on purposes, desired performance, standards, or the like, and is preferably selected from a range of 180° to 330° inclusive.

The light shielding element 22 preferably has a light reflectance of 1% or more, more preferably 30% or more, still more preferably 50% or more, even more preferably 80% or more, and desirably approaches 100%.

An inner peripheral surface of the light shielding element 22 is desirably as close to a mirror surface as possible, in order to efficiently reflect laser light. Specifically, it is preferable that surface irregularities of the inner peripheral surface of the light shielding element 22 be equal to or smaller than a wavelength of light used (i.e., laser light transmitted through the optical fiber). Here, the term “surface irregularities” refers to the difference, denoted as Rz, between the peak of the largest protrusion and the bottom of the deepest recess (maximum profile height of a contour curve: see JIS B0601).

A material for the light shielding element 22 is not particularly limited as long as the material has light shielding properties, but a material having a high light reflectance is preferable. Specific examples of the material for the light shielding element 22 include: metals such as stainless steel, aluminum, copper, and nickel-titanium (Ni—Ti) alloy; fluororesins such as polytetrafluoroethylene (PTFE); resins containing barium sulfate; reflective resins such as silicone resin; and ceramics such as alumina. Among these, from the viewpoints of light reflectance, cost, and processability, a metal is preferable, and stainless steel is particularly preferable.

A thickness of the light shielding element 22 is not particularly limited and may be appropriately selected depending on factors such as purpose, material, and mounting method. However, in consideration of functionality as a catheter, the thickness is preferably not excessive. For example, a thickness may be selected from a range of approximately 100 nm to 500 μm inclusive.

The light shielding element 22 may be fixed to an inner peripheral surface of the catheter body 21 near the distal end portion 2TE, for example, by bonding with an adhesive such as epoxy resin, by vapor deposition of a material forming the light shielding element 22, or by physical attachment using caulking or other fasteners. In the present embodiment, the light shielding element 22 is bonded with epoxy resin.

The optical fiber 3 is formed of a resin (plastic) member. The optical fiber 3 is a single-core optical fiber including: a core positioned on a center side in a radial direction; and a cladding positioned on an outer peripheral side of the core. The optical fiber 3 has a difference in refractive index between the core and the cladding in a range of 2% to 11% inclusive.

As illustrated in FIG. 1, the optical fiber 3 includes: a light transmission section 32 configured to transmit laser light from a proximal end portion (not illustrated) on the proximal side B toward the distal side T; and a light emission section 31 formed by removing a portion positioned on the outer peripheral side of the cladding within a predetermined range (near the distal end portion 3TE) in an extending direction on the distal side T. The light emission section 31 emits the laser light, transmitted through the light transmission section 32, from an outer peripheral surface.

The light emission section 31 is formed near the distal end portion 3TE of the optical fiber 3, for example, within a range of 10 mm to 30 mm inclusive from the distal end portion 3TE. The light emission section 31 can be formed by removing only the outer peripheral side of the cladding while leaving an inner peripheral side of the cladding in a thickness direction, for example, by etching.

The optical fiber 3 is inserted into the catheter 2 such that the proximal end portion 2BE of the catheter 2 and the proximal end portion (not illustrated) of the optical fiber 3 are aligned on the same side, while the distal end portion 2TE of the catheter 2 and the distal end portion 3TE of the optical fiber 3 are aligned on the same side. Then, in the axis O direction extending from the proximal end portion 2BE to the distal end portion 2TE, a light emission position (i.e., the light emission section 31) on the distal side T of the optical fiber 3 overlaps with a position of the light shielding element 22 of the catheter 2.

Near the distal end portion 3TE of the optical fiber 3, an outer peripheral surface is surrounded by the light shielding element 22 except for a partial region. A region not surrounded by the light shielding element 22 serves as an opening 23 through which light is emitted via a tube of the catheter body 21.

A laser oscillator serving as a light source connected to the proximal end portion of the optical fiber 3 on the proximal side B is configured to generate laser light and includes a semiconductor laser. The laser oscillator generates laser light by causing laser oscillation through supplying electric current to a semiconductor laser. For example, the laser oscillator generates red laser light having a wavelength in a range of 670 nm to 700 nm inclusive.

The light diffusion device 1 including the catheter 2 according to the present embodiment is used in a treatment procedure by inserting the catheter 2, in which the optical fiber 3 is inserted as described above, into a body, and guiding the distal end portion 2TE of the catheter 2 to an irradiation target (such as cancer cells). In this case, a position of the distal end portion 2TE of the catheter 2 in the body may be checked using an ultrasound or an endoscope.

After guiding the distal end portion 2TE of the catheter 2 to the irradiation target, it is necessary to orient the opening 23 toward the irradiation target. According to the light diffusion device 1 including the catheter 2 of the present embodiment, the catheter 2 in the body can be rotated by rotating the catheter 2 outside the body about the axis o, so as to adjust the position of the opening 23 in the circumferential direction. Through this operation, the opening 23 can be appropriately oriented toward the irradiation target.

In order to enable such an operation, it is necessary to impart torque transmission capability to the catheter 2 so as to prevent twisting of the catheter 2. In order to impart torque transmission capability to the catheter 2, a material having torque transmission capability may be used for the catheter 2 itself, or a member such as a torque coil for restricting rotational displacement (twisting) may be attached to an outer or inner periphery of the catheter 2. In order to identify a position of the opening 23 in the body in the circumferential direction, it is preferable to mark a specific position in a circumferential direction of the catheter 2 outside the body in advance.

After orienting the opening 23 of the catheter 2 toward the irradiation target, laser light irradiates the irradiation target to perform treatment. In the light diffusion device 1 according to the present embodiment, when the light source (not illustrated) is operated and laser light is incident on the proximal end portion of the optical fiber 3, the laser light is transmitted through the light transmission section 32 and emitted from the light emission section 31.

As described above, among light emitted from the light emission section 31, the light L1a in a direction toward the opening 23 in a circumferential direction is emitted from the opening 23 to the outside and used for irradiation. On the other hand, the light L1b, which is directed in directions within a range of a central angle θ other than the direction of light L1a in the circumferential direction, is blocked by the light shielding element 22. Accordingly, it is possible to effectively block the light Lib directed to unnecessary directions.

By using a material having a high light reflectance as the light shielding element 22, the light L1b is not only blocked, but also reflected by the light shielding element 22 and emitted from the opening 23 to the outside for irradiation. In other words, the light L1a and L1b emitted radially from the optical fiber 3 can be efficiently collected at the opening 23. Therefore, according to the present embodiment, by orienting the opening 23 of the catheter 2 toward an irradiation target in the body, it is possible to avoid irradiating non-target areas such as healthy cells, and to effectively irradiate the target.

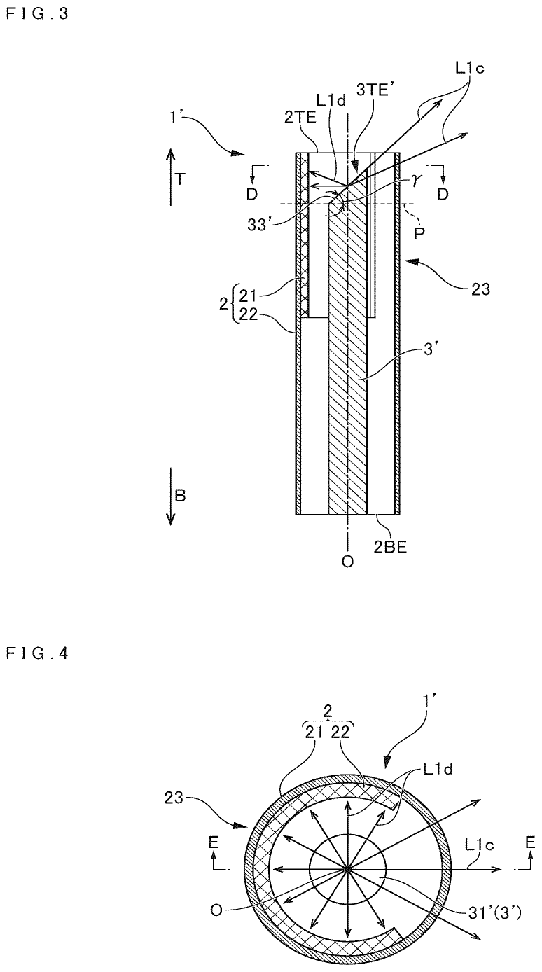

The catheter 2 according to the present embodiment can also be applied to an optical fiber whose distal end is obliquely processed. FIG. 3 is a longitudinal sectional view of a light diffusion device 1′ according to a modified example including the catheter 2 of the first embodiment; and FIG. 4 is a transverse sectional view of the light diffusion device 1′. FIG. 3 is a cross-sectional view taken along the line E-E in FIG. 4; and FIG. 4 is a cross-sectional view taken along the line D-D in FIG. 3.

As illustrated in FIGS. 3 and 4, in the light diffusion device 1′ of the present modified example, the optical fiber 3′ inserted into the catheter 2 includes an end surface 33′ at a distal end portion 3TE′, the end surface 33′ being oblique with respect to a plane P perpendicular to the axis O (i.e., the distal end is obliquely processed). At this time, an angle γ formed between the plane P and the end surface 33′ is set to be equal to or greater than an angle for total reflection of laser light transmitted through the optical fiber 3′. Specifically, assuming that a refractive index of a core of the optical fiber 3′ is n core, and a refractive index of air is n_air, the angle γ is given by γ=arcsin (n_air/n_core). When n_core=1.465, and n_air=1.0, γ becomes 43°.

An optical fiber with an obliquely processed distal end, such as the optical fiber 3′, is a probe that emits light obliquely with respect to the axis O direction (direction of the axis O) and is used for ablation treatment. The optical fiber 3′ is also considered useful as a diffuser for PDT or PIT, in which light is emitted obliquely. In such an optical fiber 3′, as illustrated in FIG. 3, a beam is split at the end surface 33′, and the split light Lid undesirably leaks in a plurality of directions other than a desired irradiation direction (direction of the light L1c).

However, by using the catheter 2 according to the present embodiment together, it becomes possible to block the split light L1d other than the intended light L1c. By using a material having a high light reflectance as the light shielding element 22, the split light Lid is not only blocked, but also reflected by the light shielding element 22 and emitted from the opening 23 to the outside for irradiation.

Therefore, according to the present modified example, even when combined with the optical fiber 3′ having an obliquely processed distal end, it is possible to effectively irradiate the target.

### Second Embodiment

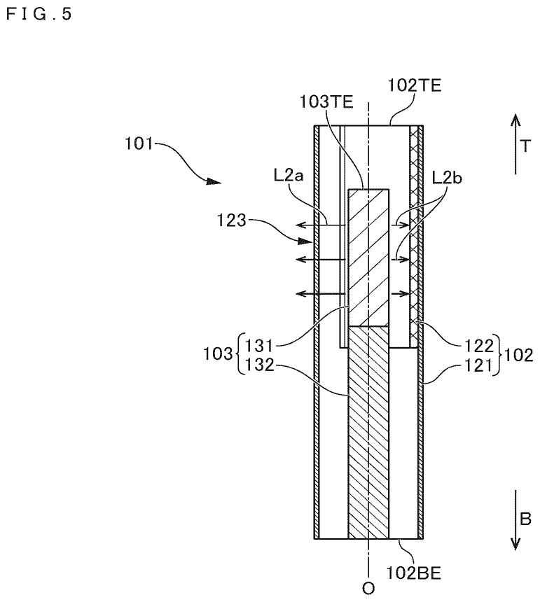

FIG. 5 is a longitudinal sectional view of a light diffusion device 101 including a catheter 102 according to a second embodiment. A transverse sectional view near a distal end portion of the light diffusion device 101 is the same as that of the light diffusion device 1 of the first embodiment; therefore, reference is made to FIG. 2.

In FIG. 5 illustrating the light diffusion device 101 including the catheter 102 according to the present embodiment, members having the same configuration as those in the light diffusion device 1 of the first embodiment are assigned three-digit reference signs having the same last two digits as those in the light diffusion device 1. Accordingly, descriptions of identical configurations are omitted, and differences will be mainly described below.

In the present embodiment, in the axis O direction, the light shielding element 122 is longer than the light shielding element 22 of the first embodiment and extends toward the distal side T. Since the light shielding element 122 is sufficiently longer than the light emission section 131 of the optical fiber 103 in the axis O direction, the light L2b directed toward unnecessary directions can be blocked even if the light spreads in the axis O direction. Therefore, it is possible to more effectively block the light L2b directed toward unnecessary directions.

It is needless to say that, by using a material having a high light reflectance as the light shielding element 122, even light such as the blocked light L2b can be reflected and guided to the opening 423. Accordingly, the light can effectively irradiate the target.

### Third Embodiment

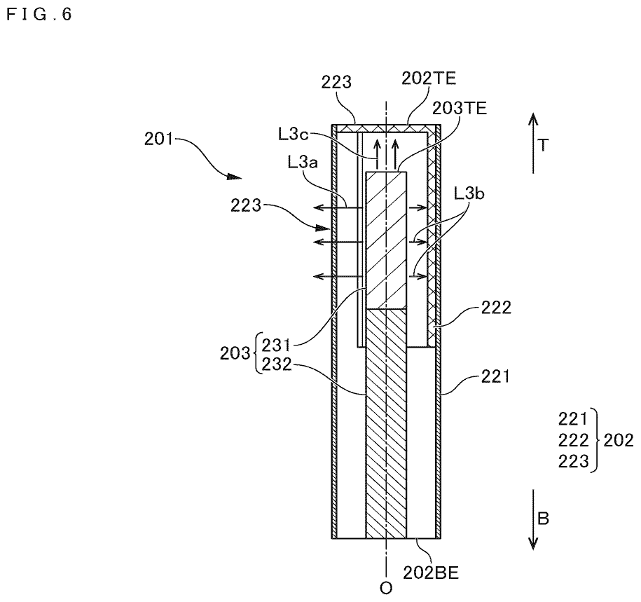

FIG. 6 is a longitudinal sectional view of a light diffusion device 201 including a catheter 202 according to a third embodiment. A transverse sectional view near a distal end portion of the light diffusion device 201 is the same as that of the light diffusion device 1 of the first embodiment (therefore, see FIG. 2).

In FIG. 6 illustrating the light diffusion device 201 including the catheter 202 according to the present embodiment, members having the same configuration as those in the light diffusion device 1 of the first embodiment are assigned three-digit reference signs having the same last two digits and a two-hundred series. Descriptions of identical configurations are omitted, and differences will be mainly described below.

In the present embodiment, in addition to a lateral light shielding element 222 having the same configuration and material as the light shielding element 22 of the first embodiment, a distal light shielding element 223 is also provided to cover a distal end portion 202TE of a catheter body 202. Both the lateral light shielding element 222 and the distal light shielding element 223 are formed of the same material and configuration as the light shielding element 22 of the first embodiment.

According to the present embodiment, the light L3c leaking from the distal end portion 203TE of the optical fiber 203 can be blocked. Accordingly, the light L3b and L3c directed toward unnecessary directions can be more effectively blocked.

### Fourth Embodiment

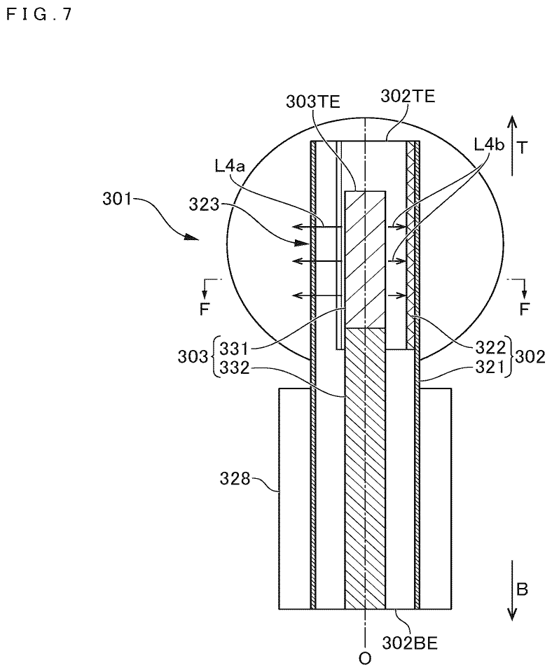

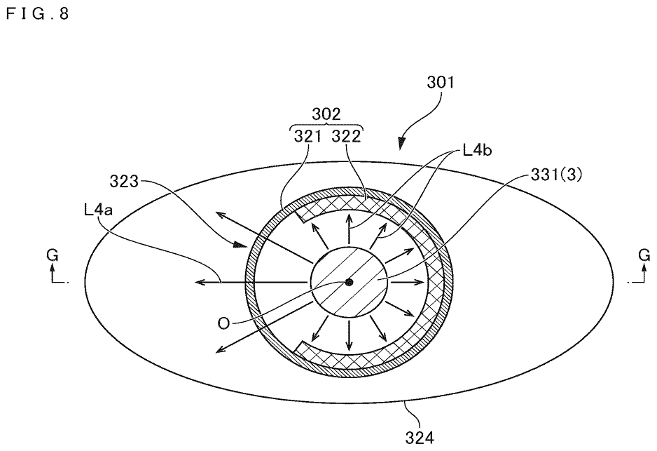

FIG. 7 is a longitudinal sectional view of a light diffusion device 301 including a catheter 302 according to a fourth embodiment; and FIG. 8 is a transverse sectional view near a distal end portion of the light diffusion device 301. FIG. 7 is a cross-sectional view taken along the line G-G in FIG. 8; and FIG. 8 is a cross-sectional view taken along the line F-F in FIG. 7.

In FIGS. 7 and 8 illustrating the light diffusion device 301 including the catheter 302 according to the present embodiment, members having the same configuration as those in the light diffusion device 1 of the first embodiment are assigned three-digit reference signs having the same last two digits and a three-hundred series. Descriptions of identical configurations are omitted, and differences will be mainly described below.

The catheter 302 according to the present embodiment is a so-called balloon catheter including a balloon 324 provided to the catheter 2 of the first embodiment. The balloon 324 is provided outside a region of a catheter body 321 in which a light shielding element 322 is provided (near a distal end portion 302TE in FIG. 7). The light diffusion device 301 according to the present embodiment further includes an endoscope 328. Specifically, an optical fiber 303 is inserted into a catheter 302, the catheter 302 is inserted into a lumen of the endoscope 328, and a portion of the catheter 302 including the balloon 324 protrudes from a distal side T of the endoscope 328. As in the case of the catheter 302 and the optical fiber 303, the endoscope 328 actually has a longer and more elongated form. Therefore, FIG. 7 is merely a schematic diagram, and the endoscope 328 extends toward a proximal side B.

In balloon catheters, it is conceivable to provide a light shielding element on an inner or outer surface of the balloon (see Japanese Patent Publication No. 2001-505443). However, since the balloon contacts the internal tissues or organs (e.g., gastrointestinal tract) in the body, there is concern that heat generated due to absorption of light from the optical fiber may affect such internal tissues or organs. Therefore, limitations may arise in treatment, such as reducing light intensity or setting an upper limit on treatment time.

In contrast, the catheter 302 according to the present embodiment includes a light shielding element 322 provided on an inner peripheral surface of the catheter body 321 and is spaced from the balloon 324. Even if the light shielding element 322 generates heat, the heat is less likely to be transferred to the balloon 324. A space is formed inside the balloon 324, and convection occurs internally, which suppresses heat generation by the light shielding element 322.

Accordingly, a temperature rise on an outer surface of the balloon 324, which contacts the internal tissues or organs, can be significantly suppressed in comparison with a case where a light shielding element is provided directly on the balloon. Thus, an influence on the inside of the body can be greatly reduced.

In comparison with a case where a light shielding element is provided on the balloon, a distance between a light emission section 331 of the optical fiber 303 and the light shielding element 322 is shorter, and therefore, a reflection efficiency of the light L4b can be increased.

In the present embodiment, a central portion in a circumferential direction of the light shielding element 322 extends toward a proximal side B in the axis O direction. The light shielding element 322 is in contact with a metal portion (not illustrated) of the endoscope 328. Therefore, heat of the light shielding element 322 can be dissipated into the metal portion of the endoscope 328. The metal portion of the endoscope 328 is elongated, thus has a large heat capacity and is expected to exhibit high heat dissipation performance. Since the metal portion of the endoscope 328 extends outside the body, heat dissipation to ambient air can also be expected.

In the present embodiment, the light shielding element 322 is extended to the proximal side B and brought into direct contact with the metal portion of the endoscope 328; however, the configuration is not limited thereto. For example, the light shielding element 322 and the metal portion of the endoscope 328 may be indirectly connected by a wire or the like made of a highly thermally conductive metal.

### The Fifth Embodiment

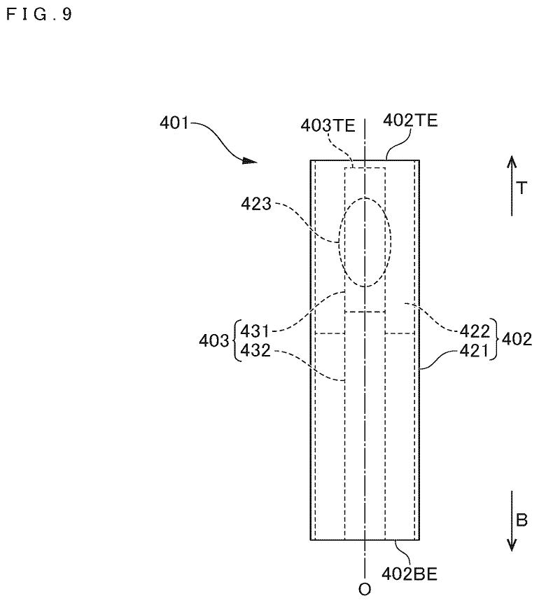

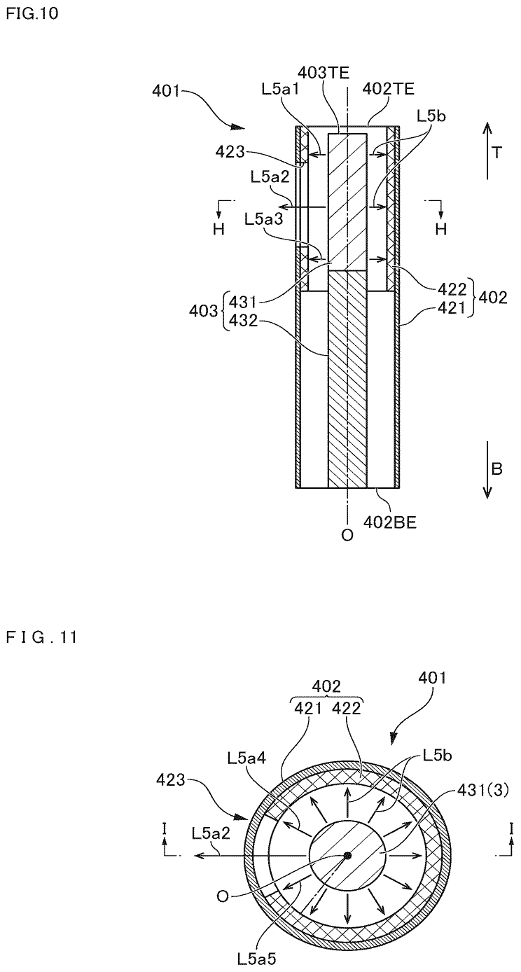

FIG. 9 is a front view of a light diffusion device 401 including a catheter 402 according to a fifth embodiment; FIG. 10 is a longitudinal sectional view of the light diffusion device 401, as viewed from the right side in FIG. 9; and FIG. 11 is a transverse sectional view near a distal end portion of the light diffusion device 401, as viewed from the same right side. FIG. 10 is a cross-sectional view taken along the line I-I in FIG. 11; and FIG. 11 is a cross-sectional view taken along the line H-H in FIG. 10.

In FIGS. 9 to 11 illustrating the light diffusion device 401 including the catheter 402 according to the present embodiment, members having the same configuration as those in the light diffusion device 1 of the first embodiment are assigned three-digit reference signs having the same last two digits and a four-hundred series. Descriptions of identical configurations are omitted, and differences will be mainly described below.

In the present embodiment, a light shielding element 422 is provided on the entire circumference of an inner surface of a catheter body 421 about the axis O extending from a proximal end portion 402BE to a distal end portion 402TE. The light shielding element 422 includes a window-like opening 423 on a lateral surface in a direction (predetermined direction) in which light is to be emitted.

In the present embodiment, only the light L5a2 directed toward the opening 423 is emitted, and the light shielding element 422 blocks not only the light L5b in non-irradiation directions in the circumferential direction, but also the light L5a4 and L5a5 that slightly deviate from the opening 423. Even when a position in the circumferential direction is the same as that of the light L5a2, the light L5a1 and L5a3, which differ in position in the axis O direction in an up-and-down manner and deviate from the opening 423, are also blocked by the light shielding element 422.

It is needless to say that, by using a material having high light reflectance as the light shielding element 422, the light such as the blocked light L5b, L5a4, L5a5, L5a1, and L5a3 can be reflected and guided to the opening 423; accordingly, the light can effectively irradiate the target.

According to the present embodiment, by appropriately designing a shape and size of the opening 423, it is possible to obtain irradiation light or an irradiated surface having a shape and size corresponding to an irradiation target. The catheter 402 according to the present embodiment is particularly useful when a microscopic region is to be irradiated.

In the present embodiment, the opening 423 has an elliptical shape, but the shape is not limited thereto. The shape of the opening may be circular, elliptical, square, rectangular, polygonal, star-shaped, or irregular. The number of openings is not limited to one and may be two or more. The shape, size, and number of openings may be appropriately designed in accordance with the irradiation target and irradiation purpose. A plurality of catheters having different shapes, sizes, and numbers of openings may be prepared in advance, and an operator may select the most suitable catheter depending on the irradiation target and irradiation purpose during treatment.

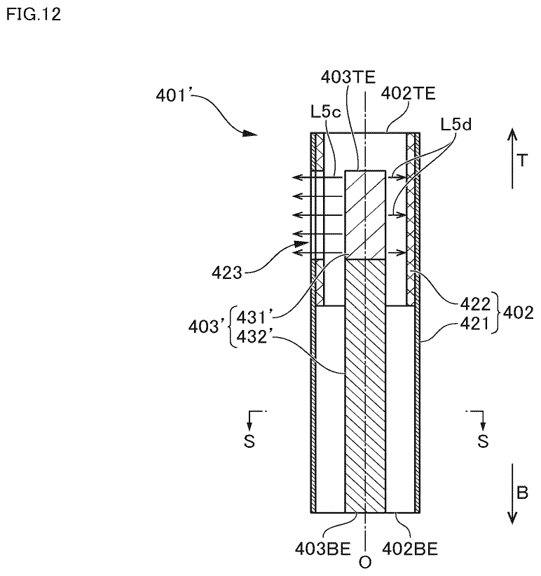

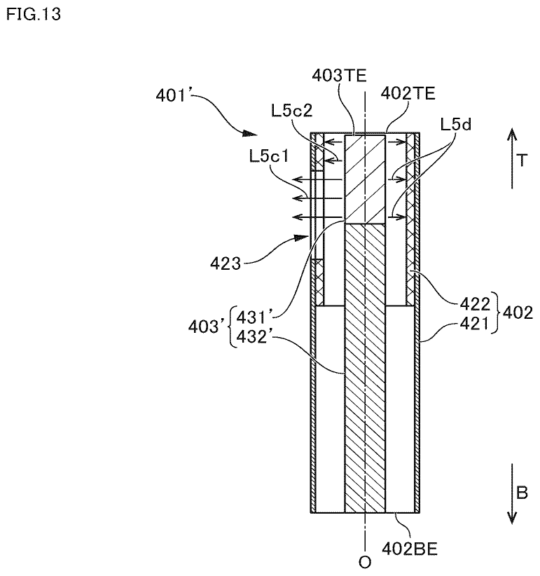

A usage example of the catheter 402 according to the present embodiment will now be described. FIGS. 12 and 13 are longitudinal sectional views of a light diffusion device 401′ including the catheter 402 according to the present embodiment. In the present usage example, an optical fiber 403′ having a shorter length in the direction of the axis O at a light emission position (i.e., the light emission section 431′) is used.

In the present usage example, the length of the light emission section 431′ in the axis O direction is shorter than the length of the opening 423. In the present usage example, the optical fiber 403′ is configured to be movable relative to the catheter 402 in the axis O direction.

FIG. 12 illustrates a state in which the entire light emission section 431′ is located within a range of the position of the opening 423 in the axis O direction. Accordingly, all light L5c directed toward the emission direction in the circumferential direction is emitted from the opening 423. Therefore, FIG. 12 represents a state in which the irradiation area is the largest (irradiation length is longest) in the present usage example.

FIG. 13 illustrates a state in which the optical fiber 403′ has been moved toward the distal side T relative to the catheter 402 from the state in FIG. 12. In FIG. 13, a distal end portion 403TE of the optical fiber 403′ is positioned close to a distal end portion 402TE of the catheter 402, and a part of the light emission section 431′ is located outside a range of a position of the opening 423 in the axis O direction.

Therefore, even the light L5c2 directed toward the emission direction in the circumferential direction is blocked by the light shielding element 422. Accordingly, FIG. 13 represents a state in which the irradiation area is smaller (irradiation length is shorter) than that in FIG. 12.

In contrast to the state in FIG. 13, when the optical fiber 403′ is moved toward the proximal side B relative to the catheter 402 from the state in FIG. 12, a state having a smaller irradiation area (shorter irradiation length) can also be obtained.

As described above, in the present usage example, the irradiation area (i.e., irradiation length) can be adjusted by appropriately moving the optical fiber 403′ toward the distal side T or the proximal side B relative to the catheter 402. During treatment, since the opening 423 serving as the light emission section is located in the body, it is preferable to preliminarily provide position markers in the axis O direction on the catheter 402 and the optical fiber 403′ outside the body, so as to enable an operator to recognize a positional relationship between the opening 423 and the light emission section 431.

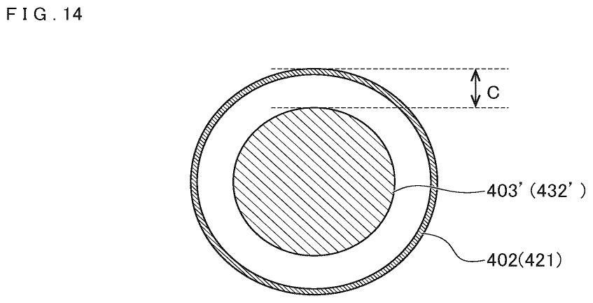

FIG. 14 is a transverse sectional view of the light diffusion device 401′ on a plane perpendicular to the axis O, and is a cross-sectional view taken along the line S-S in FIG. 12. In the present usage example, the optical fiber 403′ is appropriately moved toward either the distal side T or the proximal side B relative to the catheter 402. In order to enable such movement in the axis O direction to be performed smoothly, it is desirable that a clearance be ensured between the catheter body 421 and the optical fiber 403′.

It is preferable that a one-side clearance between the catheter body 421 and the optical fiber 403′ (432′) (one of the clearances on either side in a radial direction of the optical fiber 403′) be 5% or more of an outer diameter of the optical fiber 403′. By ensuring a sufficient clearance between the catheter body 421 and the optical fiber 403′, insufficient sliding due to friction is less likely to occur, and the optical fiber 403′ can be more easily inserted into the catheter body 421 and moved in the axis O direction.

On the other hand, although there is no particular limitation on an upper limit of a one-side clearance between the catheter body 421 and the optical fiber 403′ (432′), the clearance is preferably 20% or less of an outer diameter of the optical fiber 403′ so that the catheter body 402 does not become excessively thick. In the present usage example, for instance, the outer diameter of the optical fiber 403′ is 1 mm, and the difference between the outer diameter of the catheter body 421 and that of the optical fiber 403′ is 0.3 mm, so that the one-side clearance is 0.15 mm, which corresponds to 15% of the outer diameter of the optical fiber 403′.

It is preferable that a surface treatment for reducing friction be applied to at least one or both of the inner peripheral surface of the catheter body 421 or the outer peripheral surface of the optical fiber 403′. By applying such a surface treatment, insufficient sliding due to friction between the catheter body 421 and the optical fiber 403′ can be suppressed, and movement in the axis O direction becomes easier. Examples of the surface treatment for reducing friction include applying lubricants such as oils, powders, or chemical agents capable of imparting lubricity, and polishing or finely processing the surface.

In the present usage example, it may be difficult to grasp an inserted length of the optical fiber 403′, and it may be difficult to align a light emission section 431′ of the optical fiber 403′ with an opening 423 of the catheter 402. In order to facilitate this alignment, for example, the following three approaches (Approaches (a) to (c) ) can be employed.

Approach (a): Method using stopper

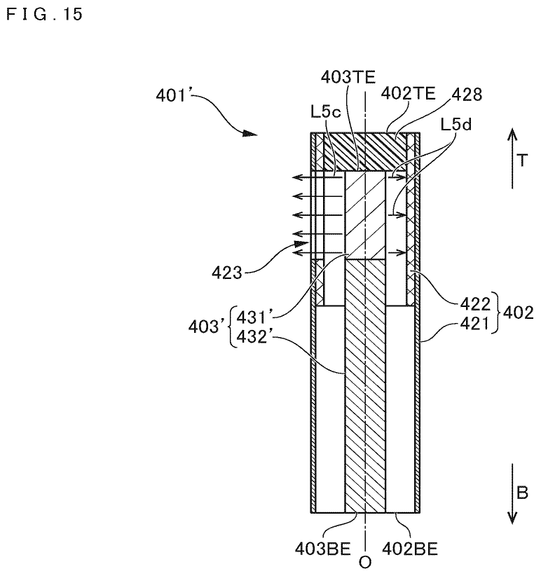

FIG. 15 is a longitudinal sectional view illustrating a modified example in which a stopper is disposed in the light diffusion device including the catheter according to the fifth embodiment. As illustrated in FIG. 15, in Approach (a), a stopper 428 is disposed inside a distal end portion 402TE of the catheter body 421.

The stopper 428 is arranged such that, when a distal end portion 403TE of the optical fiber 403′ contacts the stopper 428, the light emission section 431′ of the optical fiber 403′ aligns with the opening 423 of the catheter 402 in the axis O direction. By disposing the stopper 428, the optical fiber 403′ only needs to be inserted into the catheter 402 until the optical fiber 403′ comes into contact with the stopper 428, thereby facilitating alignment between the light emission section 431′ of the optical fiber 403′ and the opening 423 of the catheter 402.

Approach (b): Method using alignment markers

In Approach (b), a marker is provided (not illustrated) on or near a proximal end portion 403BE of the optical fiber 403′ and on or near a proximal end portion 402BE of the catheter body 421, respectively. By aligning the markers on the optical fiber 403′ and the catheter body 421, the light emission section 431′ of the optical fiber 403′ is brought into alignment with the opening 423 of the catheter 402 in the axis O direction.

By providing alignment markers, it is only necessary to align the markers of the optical fiber 403′ and the catheter body 421, so that alignment between the light emission section 431′ of the optical fiber 403′ and the opening 423 of the catheter 402 can be easily achieved. Since it is only necessary to provide alignment markers, and no additional components or design modifications are required, a configuration for aligning the light emission section 431′ of the optical fiber 403′ with the opening 423 of the catheter 402 can be established in a simple manner.

Approach (c): Method using scale markings

In Approach (c), a scale capable of checking an amount of movement of the optical fiber 403′ relative to the catheter body 421 is provided near the proximal end portion 403BE of the optical fiber 403′. The scale is provided over a predetermined length in the axis O direction, including a point within the scale at which the light emission section 431′ of the optical fiber 403′ aligns with the opening 423 of the catheter 402. It is preferable that a scale of approximately 10 cm be provided around such a point to enable fine adjustment.

As a method of providing the scale near the proximal end portion 403BE 4 the optical fiber 403′, for example, a tube with printed scale markings may be fitted over the proximal end portion 403BE of the optical fiber 403′. Alternatively, scale markings may be printed directly on the optical fiber 403′ in advance.

If a scale is provided on the optical fiber 403′, it is only necessary to adjust the position while observing the scale on the optical fiber 403′, so that alignment between a light emission section 431′ of the optical fiber 403′ and an opening 423 of the catheter 402 can be easily achieved, and fine adjustment of the position is also possible. Since it is only necessary to provide a scale on the optical fiber 403′ and no additional components or design modifications are required, a configuration for aligning the light emission section 431′ of the optical fiber 403′ with the opening 423 of the catheter 402 can be established in a simple manner.

In the present usage example, since the catheter body 421 and the optical fiber 403′ are thin and elongated, there is concern that, when the optical fiber 403′ is inserted into the catheter body 421, the optical fiber 403′ may bend or otherwise fail to maintain its orientation. In order to ensure that the optical fiber 403′ maintains its orientation without bending, the following three approaches (Approach (d) to (f) ) can be employed.

Approach (d): Increasing rigidity of optical fiber

In Approach (d), an optical fiber having high rigidity is used as the optical fiber 403′. An optical fiber having high rigidity includes an optical fiber with a large diameter or an optical fiber made of a hard material. As a specific example of the former, a plastic optical fiber (POF) having a diameter of 1 mm or more may be used. As a specific example of the latter, a quartz fiber may be used. By increasing the overall rigidity of the optical fiber 403′ and imparting stiffness thereto, it is possible to suppress bending of the optical fiber 403′ when inserted into the catheter body 421, thereby facilitating orientation retention.

Approach (e): Method using glass cap

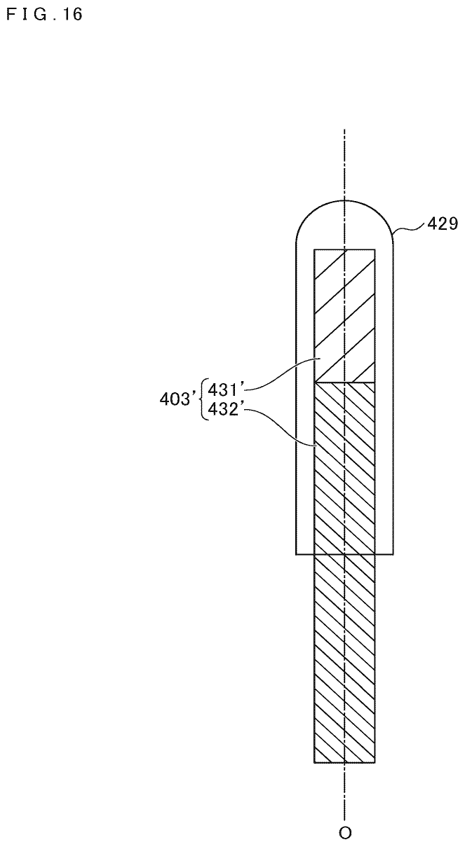

FIG. 16 is a schematic view illustrating a state in which a glass cap 428 is attached to the optical fiber 403′. As illustrated in FIG. 16, the cap 428 is a tubular glass component with a sealed distal end, and is attached so as to cover the light emission position (i.e., light emission section 431′) on the distal side T of the optical fiber 403′.

By attaching the rigid cap 428 so as to cover the light emission section 431′ of the optical fiber 403′, rigidity on the distal side T of the optical fiber 403′ can be increased and stiffness imparted. By increasing rigidity and imparting stiffness to a region near the distal end portion 403TE of the optical fiber 403′, which is susceptible to bending during insertion into the catheter body 421, bending of the optical fiber 403′ during insertion into the catheter body 421 can be suppressed, and orientation can be more easily maintained.

Since the light emission section 431′ is covered with the glass cap 428, the light emitted from the light emission section 431′ is not blocked.

Approach (f): Method using high-rigidity tube

In Approach (f), a high-rigidity tube is fitted over a whole of part of the optical fiber 403′ excluding the light emission section 431′. By covering the optical fiber 403′ with a high-rigidity tube, rigidity of the optical fiber 403′ can be increased in a region covered with the tube.

As the high-rigidity tube, a torque coil may be used. A torque coil is a metal covering (e.g., stainless steel) that imparts torque transmission capability to the optical fiber and is typically used to restrict rotational displacement (twisting) of the optical fiber. By increasing rigidity and imparting stiffness to a whole or part of the optical fiber 403′, bending of the optical fiber 403′ during insertion into the catheter body 421 can be suppressed, and orientation can be more easily maintained.

### Sixth Embodiment

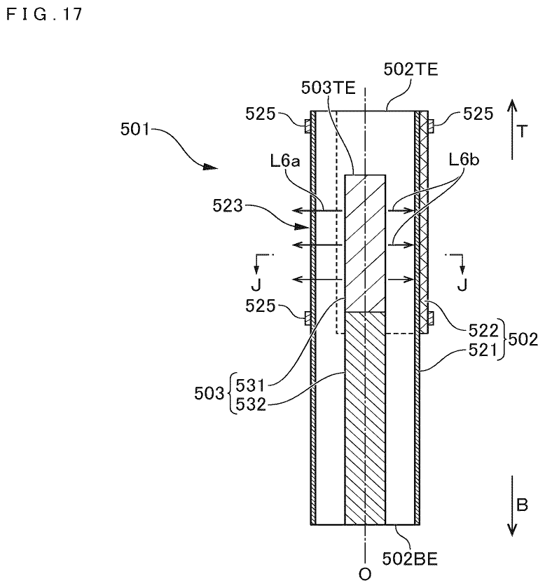

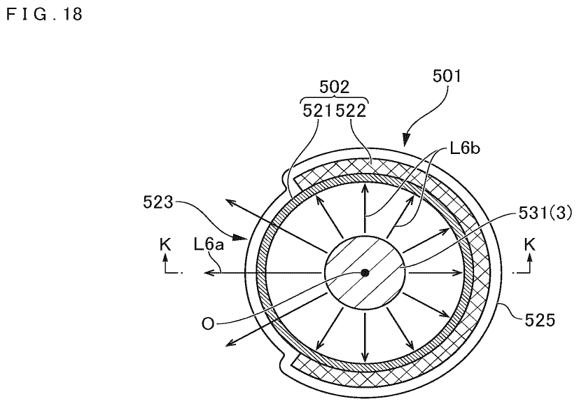

FIG. 17 is a longitudinal sectional view of a light diffusion device 501 including a catheter 502 according to a sixth embodiment; and FIG. 18 is a transverse sectional view near a distal end portion of the light diffusion device 501. FIG. 17 is a cross-sectional view taken along the line J-J in FIG. 18; and FIG. 18 is a cross-sectional view taken along the line K-K in FIG. 17.

In FIGS. 17 and 18 illustrating the light diffusion device 501 including the catheter 502 according to the present embodiment, members having the same configuration as those in the light diffusion device 1 of the first embodiment are assigned three-digit reference signs having the same last two digits and a five-hundred series. Descriptions of identical configurations are omitted, and differences will be mainly described below.

In the present embodiment, a light shielding element 522 is attached in contact with an outer peripheral surface of a catheter body 521. In the present embodiment, the light shielding element 522 is attached to the catheter body 521 by caulking, with O-rings 525, both ends in the axis O direction, or vicinities thereof, of the light shielding element 522.

The attachment of the light shielding element to the catheter body is not limited to caulking with the O-rings 525, and examples of attachment methods include, for example, using an adhesive as in the first embodiment, using other fasteners, or forming the catheter body and the light shielding element into engageable shapes and causing engagement between the catheter body and the light shielding element.

Since the catheter 502 is inserted into the body, when the light shielding element 522 is attached to the outer peripheral surface of the catheter body 521 as in the present embodiment, it is desirable to use biocompatible materials for both the light shielding element 522 and the O-rings 525. Alternatively, a layer of biocompatible material may be provided over the light shielding element 522 and the O-rings 525 so as to seal the light shielding element 522 and the O-rings 525.

### Seventh Embodiment

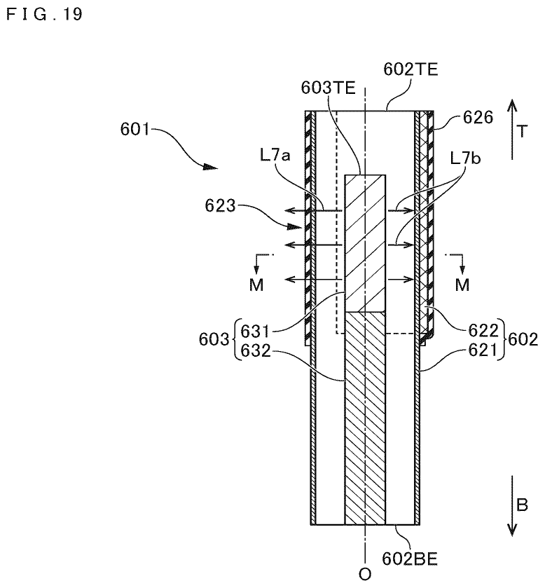

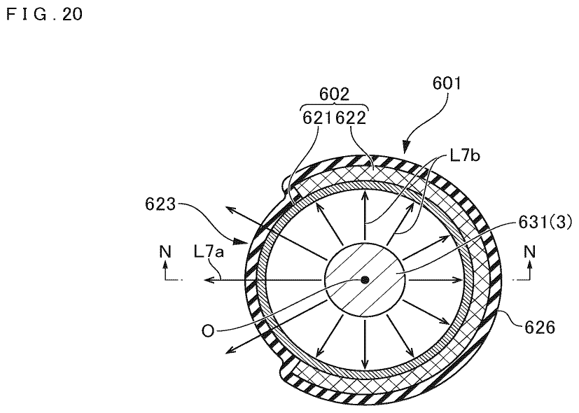

FIG. 19 is a longitudinal sectional view of a light diffusion device 601 including a catheter 602 according to a seventh embodiment; and FIG. 20 is a transverse sectional view near a distal end portion of the light diffusion device 601. FIG. 19 is a cross-sectional view taken along the line N-N in FIG. 20; and FIG. 20 is a cross-sectional view taken along the line M-M in FIG. 19.

In FIGS. 19 and 20 illustrating the light diffusion device 601 including the catheter 602 according to the present embodiment, members having the same configuration as those in the light diffusion device 1 of the first embodiment are assigned three-digit reference signs having the same last two digits and a six-hundred series. Descriptions of identical configurations are omitted, and differences will be mainly described below.

In the present embodiment, as in the sixth embodiment, a light shielding element 622 is attached in contact with an outer peripheral surface of a catheter body 621. In the present embodiment, the light shielding element 622 is attached to the catheter body 621 by covering the light shielding element 622 with a tube 626.

As the tube 626, a heat-shrinkable resin such as polyvinyl chloride may be used. The tube 626 may be placed over the light shielding element 622 and then heated to shrink, so that the light shielding element 622 is fixed by being sandwiched between the catheter body 621 and the tube 626.

As the tube 626, a material having a property of being non-transmissive to light is used. Since the portion where the tube 626 is attached is inserted into the body, it is preferable to use a biocompatible material as the tube 626. In order to protect the light shielding element 622 from body fluids or chemical solutions, it is desirable to seal the light shielding element 622 with the tube 626. A layer of biocompatible material may also be provided over the tube 6 to seal both the tube 6 and the light shielding element 622.

### Eighth Embodiment

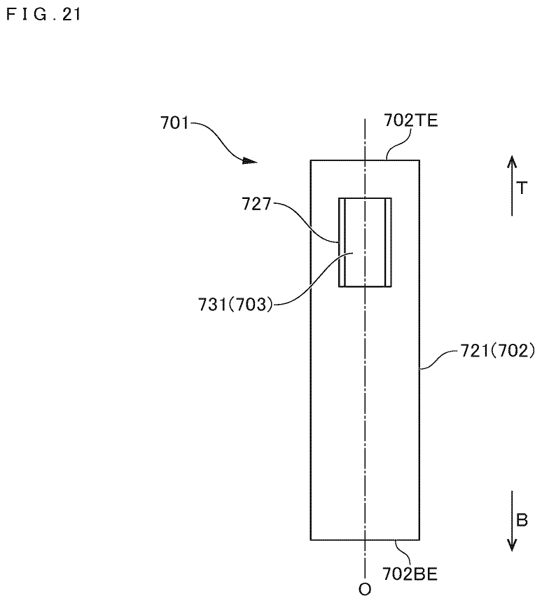

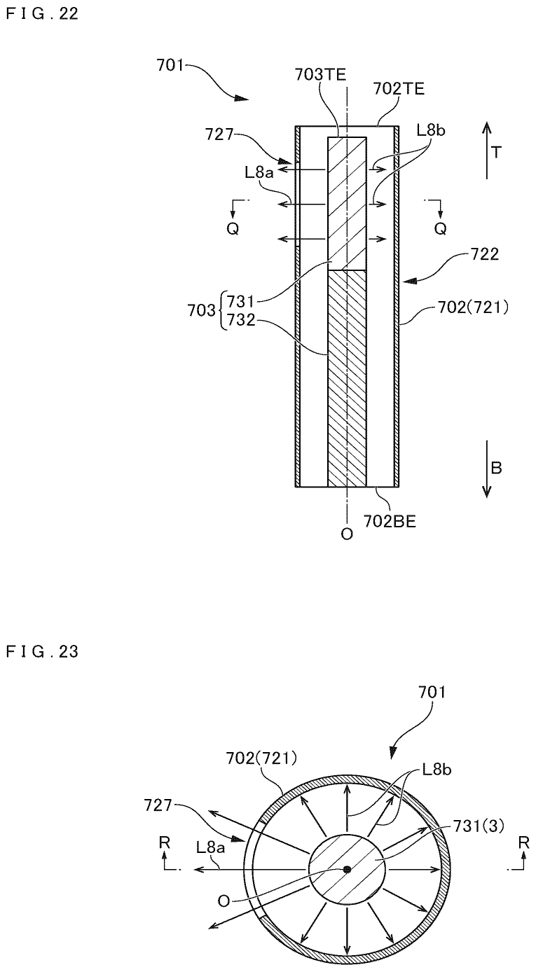

FIG. 21 is a front view of a light diffusion device 701 including a catheter 702 according to an eighth embodiment; FIG. 22 is a longitudinal sectional view of the light diffusion device 701, as viewed from the right side in FIG. 21; and FIG. 23 is a transverse sectional view near a distal end portion of the light diffusion device 701, as viewed from the same right side. FIG. 22 is a cross-sectional view taken along the line R-R in FIG. 23; and FIG. 23 is a cross-sectional view taken along the line Q-Q in FIG. 22.

In FIGS. 21 to 23 illustrating the light diffusion device 701 including the catheter 702 according to the present embodiment, members having the same configuration as those in the light diffusion device 1 of the first embodiment are assigned three-digit reference signs having the same last two digits and a seven-hundred series. Descriptions of identical configurations are omitted, and differences will be mainly described below.

In the present embodiment, the catheter body 721 has a property of being non-transmissive to light (i.e., having light-blocking properties). A catheter body 721 having a property of being non-transmissive to light can be obtained, for example, by mixing light-blocking particles into a material constituting the catheter body, by forming the catheter body as a multilayer structure in which one of the layers is composed of a light-blocking material, or by applying a coating made of a light-blocking material to an inner or outer peripheral surface of the catheter body.

The catheter 702 according to the present embodiment includes a window-like region 727 in which the catheter body 721 is not present. The window-like region 727 may be formed, for example, by cutting out a distal end portion 703TE of the catheter body 721 into a desired shape (a rectangular shape in the present embodiment).

The region 727 in which the catheter body 721 is not present functions in the same manner as the opening 423 in the fifth embodiment. A position in the axis O direction includes a region 727 in which the catheter body 721 is not present, and a region in which the catheter body 721 is present (i. e., a region other than the region 727) functions as a light shielding element (in the present embodiment, this region is referred to as “light shielding element 722”).

The catheter 702 according to the present embodiment is not provided with a separate light shielding element; and a region of the catheter body 721 including a region 727, in which the catheter body 721 is not present in a position in the axis O direction (i.e., a light shielding element 722), corresponds to the light shielding element 522 in the fifth embodiment. The window-like region 727 provided in the catheter body 721, although having a rectangular shape rather than an elliptical shape, corresponds to the opening 423 in the fifth embodiment. Accordingly, the functions and usage described in the fifth embodiment can also be similarly implemented in the present embodiment.

### Catheter Set

In the above-described embodiments of the catheter and the light diffusion device, exemplary aspects of the present invention have been described based on the configurations in which an optical fiber is inserted into a catheter to constitute a light diffusion device; however, in commercial distribution, the optical fiber and the catheter may be handled separately or as a set.

In a catheter set in which the optical fiber and the catheter are handled as a set, the optical fiber is not necessarily inserted into the catheter to constitute a light diffusion device. Rather, the optical fiber and the catheter may be distributed separately or in a bundled state. Such a catheter set is used for treatment after the optical fiber is inserted into the catheter before or during the procedure to form a light diffusion device. Accordingly, regardless of whether the optical fiber is inserted into the catheter or not, the catheter set of the present invention has the same utility and provides the same effects as the above-described embodiments of the catheter and the light diffusion device.

The embodiments described above are merely representative examples of the invention and are not intended to limit the scope of the present invention. That is, a person skilled in the art may implement various modifications without departing from the gist of the present invention, based on conventional knowledge. As long as such modifications include the configuration of the catheter of the present invention and the catheter set and the light diffusion device including the catheter, such modifications naturally fall within the scope of the present invention. For example, the configurations of each embodiment described above may be appropriately combined to implement the present invention.

### EXPLANATION OF REFERENCE NUMERALS

1, 101, 201, 301, 401, 401′, 501, 601, 701, 801: light diffusion device
2, 102, 202, 302, 402, 502, 602, 702, 802: catheter
3, 3′, 103, 203, 303, 403, 403′, 503, 603, 703, 803: optical fiber
21, 121, 221, 321, 421, 521, 621, 721, 821: catheter body
22, 122, 322, 422, 522, 622, 722, 822: light shielding element
23, 123, 223, 323, 423, 523, 623, 723, 823: opening
31, 131, 231, 331, 431, 431′, 531, 631, 731, 831: light emission section (light emission position)
32, 132, 232, 332, 432, 432′, 532, 632, 732, 832: light transmission section
33′: end surface
222: lateral light shielding element
223: distal light shielding element
324: balloon
328: endoscope
428: stopper
525: O-ring
626: tube
727: region
2BE, 102BE, 202BE, 302BE, 402BE, 502BE, 602BE, 702BE, 802BE: proximal end portion
2TE, 102TE, 202TE, 302TE, 402TE, 502TE, 602TE, 702TE, 802TE: distal end portion (catheter body)
3TE, 3TE′, 103TE, 203TE, 303TE, 403TE, 503TE, 603TE, 703TE,
803TE: distal end portion (optical fiber)

1. A catheter comprising:
a catheter body including a proximal end portion and a distal end portion; and
a light shielding element provided on the catheter body.

2. The catheter according to claim 1,
wherein the light shielding element is in contact with an inner peripheral surface or an outer peripheral surface of the catheter body.

3. The catheter according to claim 1,
wherein a balloon is provided outside the catheter body including the light shielding element.

4. The catheter according to claim 1,
wherein the light shielding element is made of metal.

5. The catheter according to claim 1,
wherein the light shielding element is formed of a reflective resin.

6. The catheter according to claim 1,
wherein the light shielding element is provided in an arc shape about an axis extending from the proximal end portion to the distal end portion of the catheter body.

7. The catheter according to claim 1,
wherein the light shielding element is provided over an entire circumference about an axis extending from the proximal end portion to the distal end portion of the catheter body, and includes an opening on a lateral surface in a predetermined direction.

8. The catheter according to claim 7,
wherein the catheter body has a property of being non-transmissive to light,
a region in which the catheter body is not present is provided,
the region in which the catheter body is not present corresponds to the opening, and a position in a direction of the axis includes the region in which the catheter body is not present, and a region in which the catheter body is present corresponds to the light shielding element.

9. A catheter set, comprising:
the catheter according to claim 1; and
an optical fiber including a proximal end portion and a distal end portion, the optical fiber being configured to emit light, which is incident on the proximal end portion, from an outer peripheral surface on a distal end side.

10. A light diffusion device, comprising:
the catheter according to claim 1; and
an optical fiber including a proximal end portion and a distal end portion, the optical fiber being configured to emit light, which is incident on the proximal end portion, from an outer peripheral surface on a distal end side,
wherein the optical fiber is inserted into the catheter, such that respective proximal end portions are aligned on the same side, and respective distal end portions are aligned on the same side, and
in a direction of an axis extending from the proximal end portion to the distal end portion, a light emission position on the distal end side of the optical fiber overlaps with a position of the light shielding element of the catheter.

11. The light diffusion device according to claim 10,
wherein the light shielding element is provided over an entire circumference about an axis extending from the proximal end portion to the distal end portion, and an opening is provided on a lateral surface in a predetermined direction, and
the optical fiber is movable relative to the catheter in the direction of the axis.

12. The light diffusion device according to claim 11,
wherein a one-side clearance between the catheter body and the optical fiber is 5% or more of an outer diameter of the optical fiber, and
a surface treatment for reducing friction is applied to at least one or both of an inner peripheral surface of the catheter body or an outer peripheral surface of the optical fiber.

13. The light diffusion device according to claim 11,
wherein a stopper is disposed inside the distal end portion of the catheter body.

14. The light diffusion device according to claim 11,
wherein a scale capable of checking an amount of movement of the optical fiber relative to the catheter body is provided near the proximal end portion of the optical fiber.

15. The light diffusion device according to claim 11,
wherein a tubular glass cap with a sealed distal end is attached so as to cover a light emission position on a distal end side of the optical fiber.

16. The light diffusion device according to claim 10,
further comprising an endoscope,
wherein the light shielding element is in direct or indirect contact with a metal portion of the endoscope.

17. A catheter set, comprising:
the catheter according to claim 2; and
an optical fiber including a proximal end portion and a distal end portion, the optical fiber being configured to emit light, which is incident on the proximal end portion, from an outer peripheral surface on a distal end side.

18. A light diffusion device, comprising:
the catheter according to claim 2; and
an optical fiber including a proximal end portion and a distal end portion, the optical fiber being configured to emit light, which is incident on the proximal end portion, from an outer peripheral surface on a distal end side,
wherein the optical fiber is inserted into the catheter, such that respective proximal end portions are aligned on the same side, and respective distal end portions are aligned on the same side, and
in a direction of an axis extending from the proximal end portion to the distal end portion, a light emission position on the distal end side of the optical fiber overlaps with a position of the light shielding element of the catheter.

19. A catheter set, comprising:
the catheter according to claim 3; and
an optical fiber including a proximal end portion and a distal end portion, the optical fiber being configured to emit light, which is incident on the proximal end portion, from an outer peripheral surface on a distal end side.

20. A light diffusion device, comprising:
the catheter according to claim 3; and
an optical fiber including a proximal end portion and a distal end portion, the optical fiber being configured to emit light, which is incident on the proximal end portion, from an outer peripheral surface on a distal end side,
wherein the optical fiber is inserted into the catheter, such that respective proximal end portions are aligned on the same side, and respective distal end portions are aligned on the same side, and
in a direction of an axis extending from the proximal end portion to the distal end portion, a light emission position on the distal end side of the optical fiber overlaps with a position of the light shielding element of the catheter.
